(19) United States
(12) Patent Application Publication
(10) Pub. No.: US 2023/0032773 A1
(43) Pub. Date: Feb. 2, 2023

(54) ANALYTE SENSING SYSTEM AND CARTRIDGE THEREOF

(71) Applicant: VSENSE MEDICAL LLC, TAIPEI CITY (TW)

(72) Inventors: CHIH-CHEN LIN, TAIPEI CITY (TW); CHIA-NAN CHIEN, TAIPEI CITY (TW); JUNG-HSIN WU, TAIPEI CITY (TW); TSUNG-CHIH HUANG, TAIPEI CITY (TW); CHIH-HAO CHU, TAIPEI CITY (TW); TIEH-KANG WU, TAIPEI CITY (TW); SHENG-YU SHIAO, TAIPEI CITY (TW)

(21) Appl. No.: 17/787,974

(22) PCT Filed: Dec. 23, 2020
(86) PCT No.: PCT/US2020/066743

§ 371 Date: Jun. 22, 2022

Related U.S. Application Data
(60) Provisional application No. 62/953,216, filed on Dec. 24, 2019.

(51) Int. Cl. B01L 3/00

(52) U.S. Cl. CPC B01L 3/502715; B01L 2300/0645; B01L 2300/0636; B01L 2300/0816; B01L 2200/0621; B01L 2400/084; B01L 2200/10

(57) ABSTRACT

A sensing system is disclosed. The sensing system includes a sensor cartridge and a readout device. The sensor cartridge includes a sensing device and a micro-channel-structure. The sensing device includes a chip member and an electrode member arranged projectively offset from each other.

### CROSS-REFERENCES TO RELATED APPLICATIONS

This application claims the benefit of U.S. Provisional Patent Application No. 62/953,216 filed on Dec. 24, 2019, which is hereby incorporated by reference herein and made as part of specification.

### BACKGROUND

### 1. Field

The present disclosure generally relates to micro-analytical sensing system, and more particularly pertains to sensing system that use sensor cartridge as a sample interface for measuring the presence or quantity of a target substance.

### 2. Related Art

The maturity of point-of-care (POC) technology would most likely stir a new wave of disruptive evolution to the field of modern healthcare. For instance, a growingly wider range of POC devices in a variety of applications have facilitated decentralization of healthcare resources and enable greater flexibility. With the advanced integration of various technical disciplines, modern healthcare devices and applications are steadily reached the multi-faced goal of predictability, reliability, rapid, portability, and cost-efficiency. For instance, readily accessible glucose meters in miniaturized shapes and forms enable diabetes patients to monitor their health conditions accurately in real-time while staying at the comfort of their homes, thereby saving patient's precious time and energy while conserving available medical resources at centralized medical institutions.

While biosensors of small form factor for POC applications are of increasing value, there have been constant challenges in the design and manufacturing of practically reliable yet affordable sensor devices. For one thing, while many places research efforts on the improvement of individual micro-electronic device fabrication, one should realize that the general design of the sensor package components is of equal importance in terms of manufacturing feasibility and device reliability.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1 illustrates a schematic application diagram of a sensing system according to some embodiments of the present disclosure;

FIG. 2A and FIG. 2B illustrate components of a sensing system according to some embodiments of the present disclosure;

FIG. 3 illustrates a perspective external view of a sensor cartridge of a sensing system according to some embodiments of the present disclosure;

FIG. 4A and FIG. 4B illustrate exploded views of a sensor cartridge according to some embodiments of the present disclosure;

FIG. 5 illustrates an exploded partial see through view of the exemplary components a sensor cartridge according to some embodiments of the present disclosure;

FIG. 6 illustrates a cross-sectional diagram of a sensor cartridge according to some embodiments of the present disclosure;

FIG. 7 illustrates a planar layout view of a sensor cartridge according to some embodiments of the present disclosure;

FIG. 8 illustrates a schematic planar view selectively focusing on two functional regions of a sensor cartridge according to some embodiments of the present disclosure;

FIG. 9 illustrates a cross sectional view of a sensor cartridge along line AA′ according to some embodiments of the present disclosure;

FIG. 10 illustrates a cross sectional view of another sensor cartridge according to some embodiments of the present disclosure;

FIG. 11 illustrates a cross sectional view showing an active chamber of a sensor cartridge according to some embodiments of the present disclosure;

FIG. 12 provides an perspective illustration of a suspended section in the micro-fluidic channel structure of a sensor cartridge according to some embodiments of the present disclosure;

FIG. 13 illustrates a cross sectional view showing a reaction chamber of a sensor cartridge according to some embodiments of the present disclosure;

FIG. 14 illustrates an exemplary sample interaction in the flow path of a sensor cartridge according to some embodiments of the present disclosure;

FIG. 15 illustrates an exemplary sample interaction in the flow path of a sensor cartridge according to some embodiments of the present disclosure;

FIG. 16 illustrates an exemplary sample interaction in the flow path of a sensor cartridge according to some embodiments of the present disclosure.

### DETAILED DESCRIPTION

The present disclosure will now be described more fully hereinafter with reference to the accompanying drawings, in which exemplary embodiments of the disclosure are shown. This disclosure may, however, be embodied in many different forms and should not be construed as limited to the exemplary embodiments set forth herein. Rather, these exemplary embodiments are provided so that this disclosure will be thorough and complete, and will fully convey the scope of the disclosure to those skilled in the art. Like reference numerals refer to like elements throughout.

The terminology used herein is for the purpose of describing particular exemplary embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” or “includes” and/or “including” or “has” and/or “having” when used herein, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

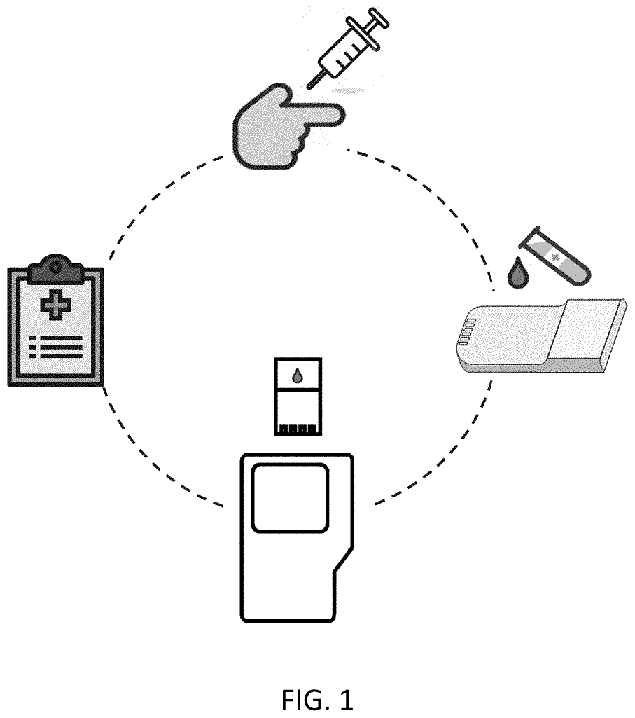

FIG. 1 illustrates a schematic application diagram of a sensing system according to some embodiments of the present disclosure.

From the top of the illustration, a sample collection process is performed. The sample collection process may take place in a public healthcare institution or a personal premise, e.g., the comfort of patient's home. The sample gathering process may involve invasive technique, e.g., blood extraction, or non-intrusive methods, e.g., throat swab, saliva or urine collection.

The application process then proceeds clockwise to the sample input stage, in which the collected sample (e.g., of bio-fluids) is provided to a sample interface component (e.g., a sensor cartridge) of a biosensor system. The sample interface component of the biosensor system may incorporate a bio-fluidic channel structure configured to guide the sample body fluid from the sample intake port to an embedded sensor component housed therein. It has been a goal for sensor device designers to provide biosensors with sufficient sensitivity to enable reliable extraction of physiological information from a sample of small size.

The process moves to the bottom of the figure to a read-out stage, in which the sample interface component is coupled to (e.g., inserted into) a read-out device of the biosensor system for the extraction of detection results. Depending on the principle of detection employed, the read-out device of the biosensor system usually having a larger size and complexity. For instance, optical-based biosensors usually call for large read-out equipment with thirsty power consumption ratings. Vibration based biosensors (e.g., atomic force microscope/AFM, crystal-quartz microbalance/QCM) require sophisticated vibration isolation arrangement, thus are not suitable for portable applications. In comparison, biosensors that incorporate modern microelectronic sensor components have been benefited from the continued advancement in micro/nano fabrication technology, which allows the reduction of form factor in not only the sample interface components of the biosensor system, but also the read-out device itself. In some applications, the read-out device of the biosensor system is integrated into a portable unit, as illustrated in the drawings.

The sample diagnosis process then proceeds to the result generation stage. With the sophistication of microelectronic sensor components, detection accuracy has been improved to meet practical application requirements, and the result turnaround time has been significantly reduced (e.g., in the matter of hours). Moreover, advanced micro/nano-fabrication technology enables predictable and reliable batch production of the sample interface components, thus helps to reduce unit cost and makes disposable sensor component a viable reality. Owing to the disposable and dynamic nature of the sensor employment, the diagnosis process may be relatively stakelessly repeated as practical application requires.

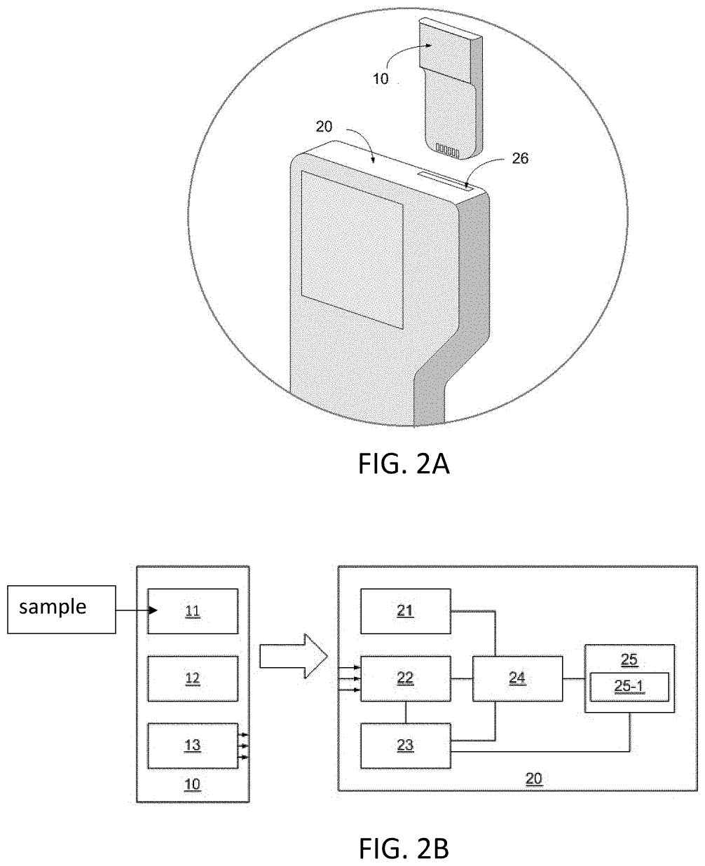

Referring concurrently to FIG. 2A and FIG. 2B, which illustrate components of a sensing system according to some embodiments of the present disclosure. For one thing, FIG. 2A illustrate hardware components of an exemplary biosensor system, while FIG. 2B illustrates a schematic block diagram of exemplary functional components of a sensing system according to some embodiments of the present disclosure.

The exemplary sensing system includes a sensor cartridge 10 and readout device 20. In some embodiments, the sensor cartridge 10 serves as a sample interface configured to receive extracted physiological fluid sample. The sensor cartridge 10 may be provided with a micro-channel-structure 11 arranged to receive and guide the input sample fluid to a sensing device 12 that includes microelectronic sensor components, and I/O ports 13 configured to interface with the readout device 20 for information extraction. In some embodiments, the readout device 20 is provided with a fluid driving module 21 configured to induce fluid flow in the micro-channel structure 11 of the cartridge 10, I/O ports 22 for interfacing the cartridge I/O ports 13, a readout module 23 that comprises electronic readout circuitries, a power module 24, and an output module 25 for outputting detection results.

In some embodiments, the output module 25 includes display unit 25-1 configured to present audio/visual information of the detection results in user comprehensible format. In some embodiments, the fluid driving module 21 includes hardware arrangements configured to drive fluids, e.g. sample fluid, within the micro-channel-structure 11. For example, the sample fluid may include a target substance, such as an analyte, whose presence or quantity (e.g., concentration) is to be determined. The fluid driving module 21 may incorporate off-cartridge motor and pumping components arranged to induce fluid flow in the cartridge, so as to transport analyte to the sensing surface(s) of the cartridge sensing components (e.g., device 12). The off-board fluid driver arrangement may enable further miniaturization of the cartridge design.

In some embodiments, the readout module 23 comprises application specific circuit components designed to detect and convert variations in target analyte concentration into an electrical signal, such as, current, voltage, capacitance, resistance, etc. In some embodiments, the power module 24 is provided with A/C power interface for long operational duration, or with D/C power source for portability.

As shown in FIG. 2A and FIG. 2B, the exemplary readout device 20 is provided with an insertion slot 26 configured to at least partially receive the sensor cartridge 10. Upon insertion of the sensor cartridge 10 into the readout device 20, the respective I/O ports 13 and 22 may establish signal connection, while the fluid driver module 21 may establish mechanical engagement with a portion of the micro-channel-structure 11, so as to exert driving force that induce fluid flow in the micro-channel(s).

In some embodiments (as will be illustrated with further detail in the later section), the micro-channel structure is provided with a sample intake port along with one or more on-board fluid reservoirs (in which various functional fluids, e.g., buffer/wash fluids may be sealingly stored). The fluid driver module 21 may include a pump (such as a displacement pump) configured to engage the micro-channel-structure 11 to induce flow of the fluids in the flow paths defined in the micro-channel structure 11. The length of the flow path and the flow rate of fluids in the micro-channel-structure 11 may be setup in accordance with suitable time duration for a particular testing procedure.

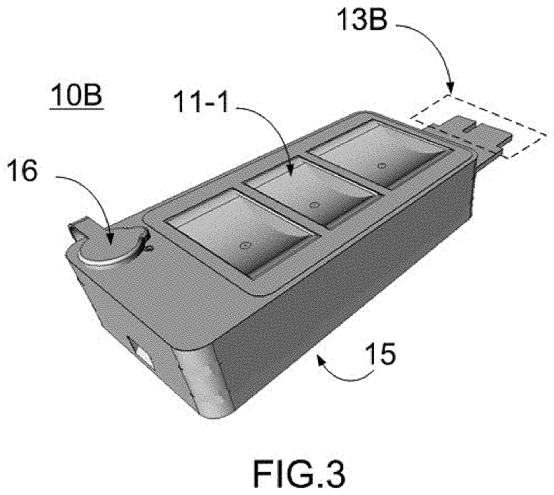

FIG. 3 illustrates a perspective external view of a sensor cartridge of a sensing system according to some embodiments of the present disclosure.

The exemplary sensor cartridge 10B comprises a housing 15 and a I/O interface 13B. In some embodiments, the housing 15 may comprise several layers of sub-members, in which a micro fluid channel structure is defined and micro-electronic sensor components are enclosed. In some embodiments, the electronic sensor components are provided on a mounting surface of a substrate (e.g., a PCB), while a majority portion of the substrate is enclosed in the housing 15. The substrate provides mechanical support as well as electrical interconnection between various sensor components. In the illustrated embodiment, an exposed portion of the substrate (e.g., the portion shown in the dotted box) protrudes from one end of the housing to host the I/O interface 13B.

The housing 15 may be provided with additional micro-channel related components at externally accessible locations. For instance, an inlet cap 16 is arranged over a sample inlet of the micro-channel structure to prevent input sample fluid from spilling. Moreover, one or more onboard fluid reservoirs 11-1 may be provided at a top level of the housing 15 to allow mechanical manipulation by a fluid driving mechanism (e.g., driving module 21) from a read-out device. In the illustrated embodiment, the housing 15 is provided with three troughs arranged in tandem along the longitudinal axis thereof. The troughs are configured to store functional fluids (e.g., buffer solution, wash fluid, reaction fluid, etc.) with predetermined volume, which are sealed by a flexible membrane over the top housing surface. Each trough of the onboard fluid reservoir 11-1 is accessible by a micro-channel (e.g., as shown at the bottom center portion of the trough), so as to enable the stored fluid to be driven into the micro-channel structure embedded in the housing 15 upon exertion of force.

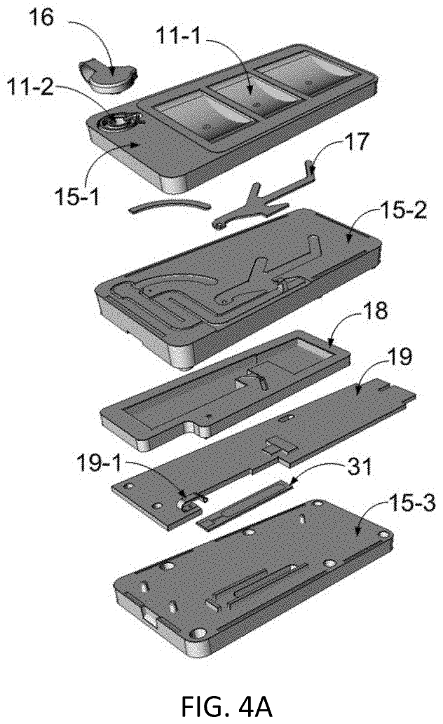

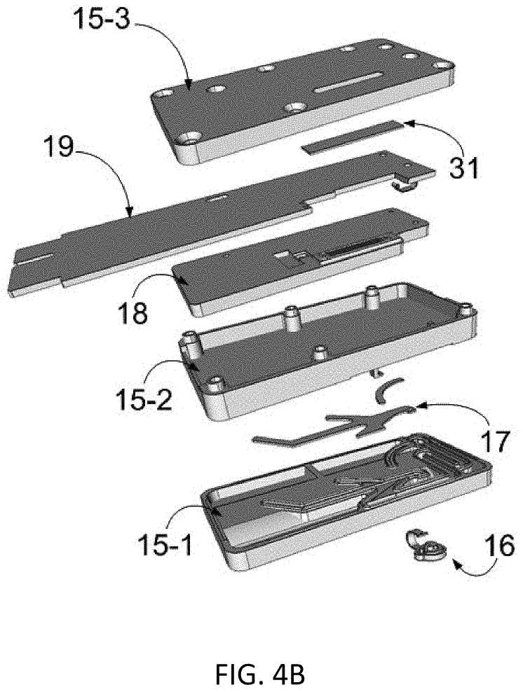

FIG. 4A and FIG. 4B illustrate exploded views of an exemplary sensor cartridge according to some embodiments of the present disclosure. Specifically, FIG. 4A shows an exploded perspective view of the cartridge components from an upper point of view (e.g., over the top surface of the cartridge fluid reservoirs), while FIG. 4B takes a lower point of view showing the bottom side of an exemplary sensor cartridge.

The exemplary sensor cartridge includes a top layer member 15-1 on which a sample intake port 11-2 and one or more fluid reservoir 11-1 are accessibly arranged; a middle layer member 15-2 in which a network of micro-fluidic trenches are formed; a substrate 19 that provides mechanical support for necessary electronic sensor components; a lower channel layer 18 configured to be fitted between the mounting surface of the substrate 19 and the middle layer member 15-2 to form a fluid-tight lower level flow path that guides sample fluid toward the sensor components on the substrate 19; and a bottom layer member 15-3 configured to engage the bottom face of the substrate 19.

In the illustrated embodiment, top layer member 15-1 and the middle layer member 15-2 are formed with micro-fluidic trench patterns on the top and bottom faces thereof. The micro trench patterns may be aligned to each other upon coupling of the top and middle members 15-1, 15-2 and cooperatively form an upper level of the micro-channel structure. In the illustrated embodiment, the upper level channel structure (which makes up a portion of the micro-channel structure) includes the sample intake port 11-2 (which is configured to be sealed by a cap member 16), the fluid reservoirs(s) 11-1, and the interconnecting channel network underneath the intake port and the reservoir(s). In the illustrated embodiment, the micro-fluid channel structure is formed from several layers of horizontal members to maintain manufacturing simplicity, as the formation of a sophisticated, multi-level channel network in a unitary bulk structure may be unrealistic in terms of mass production feasibility. In the illustrated embodiment, the top and middle members 15-1, 15-2 are further formed with a substantially hollow body to save weight and material cost.

In some embodiments, the upper and the middle layer members 15-1, 15-2 are made from relatively rigid plastic material(s), e.g., Polypropylene, Polycarbonate, and acrylonitrile butadiene styrene/ABS. The harder plastic material in the stacked layer members (e.g., members 15-1, 15-2, 15-3) may allow their rigid exposed surfaces to cooperatively provide structural protection for the internal cartridge components, thereby eliminating the need for additional housing member. For example, the exemplary cartridge in the instant embodiment utilizes a housing (e.g., housing 15 shown in FIG. 3) formed from the outer surfaces of the stacked layer members (15-1, 15-2, 15-3), thus effectively saves volume, weight, and structural complexity.

In some embodiments, selective portions of the micro-channel trench (e.g., the portion formed between the upper and middle members 15-1, 15-2) may be provided with additional fluid sealing features (e.g., gasket 17) to ensure better fluid sealing properties. In some embodiments, the gasket 17 may be shaped to conform to a particular segment of the channel pattern. In some embodiments, the gasket is made from softer material(s), e.g., rubber and silicone.

In the illustrated embodiment, the substrate 19 has a mounting surface (e.g., the surface that faces toward the lower channel layer 18) that hosts one or more micro/nano-electronic components. The electronic components may include semiconductor based microchip with biosensor components integrated thereon. The biosensor components may include special types of field effect transistors (FET), such as ion-sensing field effect transistors (ISFET) or extended gate field effect transistors (EGFET). The bio-sensor chip may be disposed over the mounting surface of the substrate 19 through suitable surface mounting techniques, such as wire bonding or flip chip arrangements. The sensing surface (e.g., a first sampling surface) of the microchip is upwardly arranged to face the lower channel layer 18, thereby allowing the integrated electronic sensor components to gain fluid access.

The substrate 19 may include a printed circuit board (PCB) such as Single-layer PCBs, Double-layer PCBs, Multi-layer PCBs, Rigid PCBs, Flexible PCBs, Rigid-Flex PCBs, High-frequency PCBs, Aluminum-backed PCBs. In the illustrated embodiment, the substrate 19 is provided with a notch (where an electrode contact 19-1 is located). In the illustrated embodiment, the notch is provided to accommodate an electrode member 31 (on which forms a second sampling surface) in a low-profile configuration. The electrode member 31 may be configured as an extended gate in an EGFET application, or a reference electrode in an ISFET application. In such a low-profile configuration, an electrode contact 19-1 is provided at an edge region of the notch to enable electrical connection between the on-board sensor components and the electrode member 31. Nevertheless, in some embodiments, the electrode member may be provided over the mounting surface of a substrate instead (e.g., formed as a plated conductive region over the mounting face of a substrate that does not have a notch profile).

In the illustrated embodiment, the lower channel layer 18 is configured to establish direct contact with the mounting surface of the substrate 19. In some embodiments, the lower channel layer 18 is formed from elastomeric material having a relatively low Young's modulus (i.e., softer than the upper/middle layer members 15-1, 15-2). The lower channel layer 18 is provided with micro trench patterns that forms a lower level of the micro-fluid channel structure upon assembly over the mounting face of the substrate 19. The lower level channel structure is configured to guide the sample/functional fluid over the sensing surfaces of the electrode member 31 or the micro sensor chip on the substrate 19. In some embodiments, the lower level channel structure is arranged to sequentially guide the sample fluid over the sampling surfaces of the electrode member 31 and the onboard sensor chip (not explicitly labeled), and finally toward a waste gathering compartment (not explicitly labeled). The order of the flow sequence over the first and second sampling surfaces (e.g., of the microchip and the electrode member, respectively) need not be limited to that shown in the illustrated figures, as long as the lower level channel structure allows the first and the second sampling surfaces to be kept with a projective offset at a predetermined planar separation. In some embodiments, a lateral separation between the micro sensor device and the electrode member 31 is no less than 0.1 mm.

As illustrated in this embodiment, the structurally separable lower channel layer 18 made from softer material may offer enhanced fluid sealing capability over the mounting surface of the substrate 19. Moreover, from a device packaging perspective, the standalone design of the lower channel member 18 enables higher degrees of practical flexibility in terms of manufacturing tolerance. By way of example, the separable lower channel layer 18 may better accommodate the height variation of various surface mounted components while providing better fluid sealing at the hetero-interface between package components of the cartridge, thereby ensuring operational reliability and extending shelf-life of the sensor device.

In the illustrated embodiments, the lower channel layer 18, the substrate 19, and the electrode member 31 are disposed between the middle layer member 15-2 and the bottom layer member 15-3. When the middle layer member 15-2 and the bottom layer member 15-3 are mechanically coupled to each other, a compression force is applied to the lower channel layer 18 and the substrate 19 to form a mechanical seal there-between. Meanwhile, a mechanical force is applied to the connector 19-1 to establish electrical coupling between the electrode member 31 and the substrate 19.

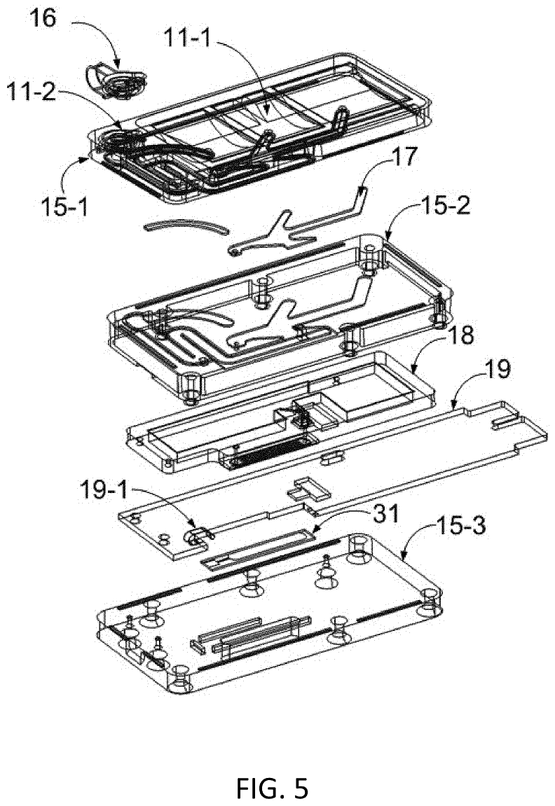

FIG. 5 illustrates an exploded partial see-through view of the components an exemplary sensor cartridge according to some embodiments of the present disclosure. The partial see-through diagram provides a better view at the arrangement of an exemplary micro-channel structure otherwise embedded in the various structural members of the sensor cartridge components. For the ease of comprehension, the exemplary biosensor cartridge in FIG. 5 resumes comparable component arrangements and identical element label numerals.

As can be better seen from the see-through illustration, the exemplary top layer member 15-1 is provided with fluid reservoir features (e.g., tank 11-1) and sample intake port (e.g., inlet 11-2) on one face thereof, while having a variety of micro-channel trench features formed on the opposite face. Likewise, the upward-facing surface of the middle layer member 15-2 is provided with micro-channel trench features that correspondingly match the trench pattern of the upper member 15-1. In this way, the half-open micro-channel trench features from different layer members may cooperatively form an enclosed micro-channel network upon coupling of the package components.

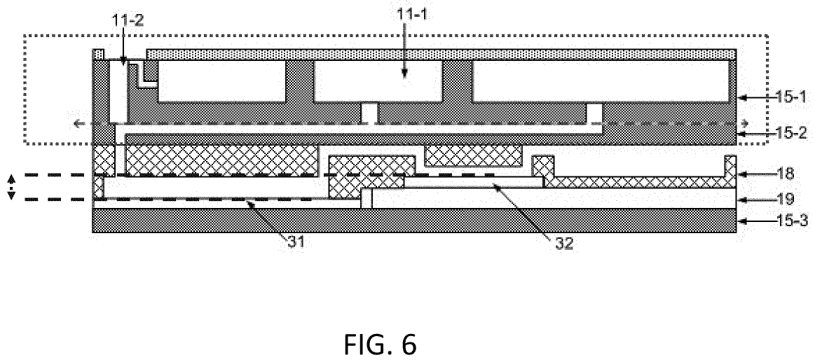

Referring concurrently to FIG. 6, which illustrates a cross sectional diagram of an exemplary sensor cartridge according to some embodiments of the present disclosure. The cross sectional illustration provides a better visualization of the embedded multi-level micro channel structure over the substrate's onboard and off-board sensor components (e.g., the onboard sensor chip 32 and the off-board electrode member 31).

The upper level of the micro channel network (e.g., the upper portion of the channel structure enclosed in the dotted box of FIG. 6) may be formed in/between the upper layer members (e.g., the layer members 15-1, 15-2) upon assembly. It should be noted that the exemplary micro channel arrangement shown in the instant drawings is mainly for illustration purpose; the actual channel network layout may be otherwise designed for specific application requirements.

The coupling between the layer members may be achieved through fluid sealing arrangements, e.g., water resisting adhesives or tapes. In some embodiments, the channel-housing components (e.g., layer members 15-1, 15-2) are made of similar/identical material (e.g., molded thermoplastic), and the package components are coupled to each other using low temperature, permanent joining techniques such as ultrasonic welding or laser welding. In such embodiments, the upper level channel structure (which may include, e.g., the sample inlet 11-2, the fluid reservoir 11-1, and the vertical/lateral extending conduits there-under) may be formed in a substantially water-tight manner. An observable welding interface may be generated between the cartridge sub-members. In some embodiments, the package components of the cartridge (e.g., the layer members 15-1, 15-2, etc.) may possess substantially hollow construction, thereby enabling weight-saving and material conservation.

Similarly, the lower channel layer 18 is provided with embedded micro conduit features designed to form the lower level of the channel structure upon assembly of the package components. By way of example, the lower channel layer 18 may be made from a bulk of softer or elastic material (e.g., silicone) with various chambers and conduit features (e.g., via and trenches) defined therein. For instance, a first chamber (e.g., a reaction chamber) may be formed over the sampling surface of electrode member 31, while a second chamber (e.g., an active chamber) may be formed over the sampling surface of the sensor chip 32 on the substrate 19. In the embodiment shown in FIG. 6, the lower level channel structure further comprises a third chamber (e.g., a waste gathering chamber) downstream (e.g., to the right of the page) of the second chamber.

Conduit features having narrower width (from a planar perspective) that traverses across different heights (i.e., elevation in a lateral cross section such as shown in FIG. 6) may be provided between the chambers to enable fluid communication there-between. As illustrated in FIG. 6, the micro conduit feature between the first and the second chamber has an inversed U-shape profile. From the illustrated point of view, the inter-chamber conduit feature comprises a pair of vias with unequal lengths (e.g., unequal vertical segments) and a suspended lateral segment traversing between the vias. By way of example, the suspended section of the micro-channel-structure is arranged at an elevation higher than an immediate upstream section thereof (e.g., the section of the micro-channel over the electrode member 31).

From a sampling efficiency perspective, the none-overlapping, streamline flow path created by the suspended section between the first chamber (e.g., over the electrode member 31) and the second chamber (e.g., over the sensor chip 32) may reduce flow turbulence and maintain inter-channel fluid pressure, thereby increasing sampling efficiency over the sensing surfaces. On the other hand, from a packaging aspect, the suspended overpass arrangement in the lower channel layer 18 provides the exemplary micro-channel structure a higher degree of accommodation for the step/height variation among circuit components over/around the substrate 19, thereby increasing manufacturing tolerance and device reliability.

As illustrated in the instant embodiment, the sensing surface of the electrode member 31 (indicated by the lower dotted line) is arranged at a lower elevation than the elevation of the sensor chip 32 over the substrate 19. The increased tolerance for height variation in turn increases design flexibility. For one thing, the lower placement of the electrode member 31 allows the reduction of overall device thickness, at the same time enables the utilization of larger electrode size (i.e., larger capturing surface over the electrode member) while maintaining sufficient clearance in the corresponding reaction chamber.

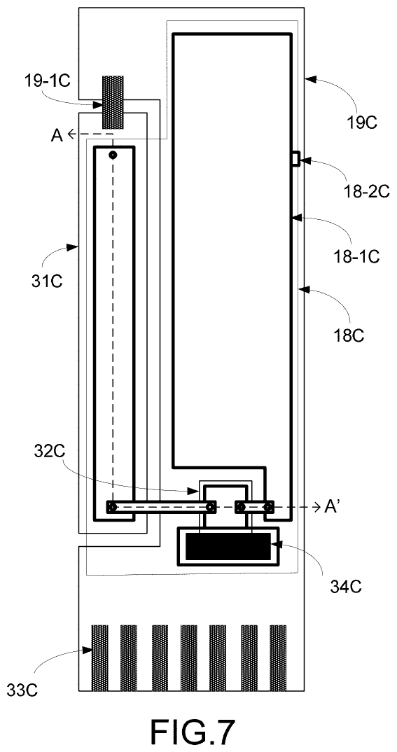

FIG. 7 illustrates a planar layout view of a sensor cartridge according to some embodiments of the present disclosure. For one thing, the schematic planar view of FIG. 7 shows the placement of an electrode member (e.g., reference electrode 31C) and a lower channel layer (e.g., member 18C) with respect to a substrate (e.g., PCB 19C) upon assembly. For instance, the plan view may reflect the layout from the horizontal plane represented by the dotted line over the sensing surface of the sensor chip 32 as shown in FIG. 6).

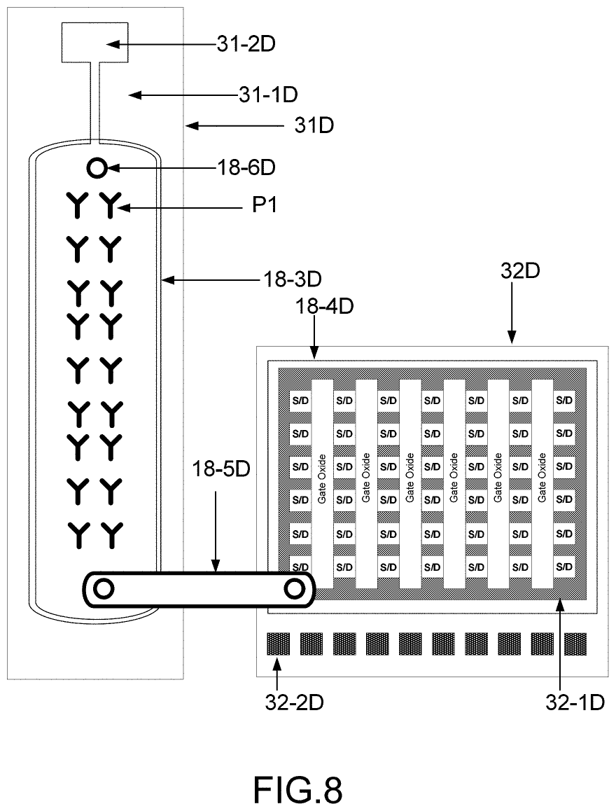

The exemplary sensor cartridge comprises a sensing device that includes, among other things, a chip member 32C and an electrode member 31C. The chip member 32C may be disposed over the mounting surface of the substrate 19C, with its active surface (i.e., sensing surface where the micro sensor components are hosted) arranged upward toward the active chamber defined in the lower layer member 18C. The active surface may comprise the components of various micro-electronic devices, e.g., the source and drain regions of a bio-sensing FET. One or more micro (or even nano) sensing element may be provided over the active surface. In some embodiments, an array of multiple micro-sensor elements is provided (as illustrated in FIG. 8) to improve detection sensitivity/accuracy. The region of the active surface exposed to the micro-channel structure (e.g., accessible from the active chamber defined in the lower channel layer 18C) defines a first sampling area.

In the illustrated embodiment, the electrode member 31C servers as a reference electrode for an ISFET-based biosensor device. The upward facing surface (i.e., the sample interface) of the electrode member 31C is specially treated, e.g., provided with proper coating, on which suitable bio-sensing probes (e.g., ligand/antibody specific to a target substance in an analyte) are immobilized, thereby forming a capture surface. The region of the capture surface exposed to the micro-channel structure (e.g., accessible from the reaction chamber formed over the electrode member 31C) defines a second sampling area.

In the illustrated embodiment, the active surface of the chip member 32C is arranged projectively offset the capture surface of the electrode member 31C. The planar offset layout of the chip member 32C and the electrode member 31C (where each of the respective sampling surface is provided with an individual sampling chamber) helps to increase the sensor device's detection accuracy while maintaining small-form factor of the overall package size. For one thing, modern fabrication technique allows the provision of miniaturized electronic sensor components on a sophisticated integrated circuit chip (e.g., chip member 32C). The small size of the sensor chip (e.g., chip member 32C) calls for lower degree of accommodation in a sensor device, thereby increasing packing flexibility. On the other hand, higher detection accuracy may be obtained by the utilization of larger capturing interface on the electrode member (i.e., larger sensing surface in contact with analyte). The structurally separated electrode member (e.g., electrode 31C, which may be configured to function as an extended gate for an EGFET based sensor, or a reference electrode for an ISFET based sensing device) may be designed to possess a sampling area a magnitude larger than the allowed sensing area over a micro-sensor chip, while being placed at a practically feasible location in the in the sensor package.

The exemplary electrode member 31C utilizes a structurally separated arrangement detachable from the substrate 19C. In some embodiments, a projected planar offset between the active surface and the capture surface is kept at a distance of no less than 0.1 mm. In the illustrated embodiment, the stand-alone electrode member 31C is placed in a notch profile provided at one side (e.g., left hand side as shown in FIG. 7) of the substrate 19C. The exemplary electrode member 31C is provided with an elongated rectangular profile that maintains geometric simplicity while forming a prolonged sample-interfacing path with an incoming analyte from the micro-channel structure. The off-board placement of the electrode member in the notch feature of the substrate may further facilitate thickness reduction of the device package.

In addition, as the lower channel layer 18 is configured to establish fluid flow path across the respective sampling surfaces on the electrode member 31C and the chip member 32C, and a planar coverage thereof extend beyond the mounting surface of the substrate (e.g., over the notched profile of the substrate).

Connector 19-1C is disposed on the substrate 19C at a periphery of the notch profile to enable electrical coupling between the substrate 19C and the electrode member 31C. Moreover, a plurality of contact pads 33C are formed at one end of the substrate 19C (e.g., the end that faces the bottom of the page in FIG. 7) to serve as the input/output interface (e.g., I/O ports 13 depicted in FIG. 1) between a sensor cartridge (e.g., cartridge 10 depicted in FIG. 1) and a readout device (e.g., readout device 20 depicted in FIG. 1). In some embodiments, the provision of onboard I/O interface on a substrate with sufficient mechanical rigidity helps to reduce package complexity while ensure device reliability and durability.

In some embodiments, the first sampling area and the second sampling area are of substantially different dimensions. In some embodiments, the second sampling area of the electrode member 31C is substantially larger than the first sampling are of the chip member 32C. For instance, a ratio of the first sampling area and the second sampling area is substantially less than 1. In some embodiments, the ratio between the first sampling area and the second sampling area is in a range of about 1×10⁻⁸ to about 1.

The onboard microchip (e.g., chip member 32C) may be provided over the substrate surface through suitable surface mounting technology, e.g., flip chip or wire bonding techniques. In the illustrated embodiment, the exemplary chip member 32C configured to have its electrical interface (e.g., I/O pads) arranged along only one of its four edges (e.g., the edge shown toward the bottom of the page in FIG. 7). In the illustrated embodiment, the plurality sides (or edges) of the chip member that are free from electrical interfaces form a plurality of free edges, which in turn provide the sensor chip enhanced fluid access from the micro-channel structure arranged there-above. Meanwhile, encapsulation 34C is disposed along only the bottom edge/side of the exemplary chip member 32C to provide protection for the electrical connection between the chip and the substrate (e.g., the pads and wires) from moisture and mechanical stress.

In some embodiments, a waste chamber 18-1C and an air vent 18-2C of the micro-channel structure may be formed in the lower channel layer 18C. The waste chamber 18-1C is shown to be arranged downstream of the sampling chambers and configured to collect excessive substances provided during testing procedure. The air vent 18-2C is configured to regulate the pressure within the micro channel structure.

FIG. 8 illustrates a schematic planar view selectively focusing on two functional regions of a sensor cartridge according to some embodiments of the present disclosure. For instance, FIG. 8 provides a schematic illustration on the micro sensing components (e.g., non-observable to naked human eyes) on the respective sampling surfaces of the exemplary electrode member 31D and the exemplary chip member 32D. In the illustrated embodiment, an exemplary electrode member 31D is provided with a base body 31-1D and a coating layer 31-2D disposed on the channel-facing side thereof (i.e., the side that's visible on the page of FIG. 8) to form a capture surface. Moreover, in the illustrated embodiment, the capture surface of the electrode member is provided with an array of capture probes P1 immobilized over the coating layer 31-2D on the base body 31-1D.

For one thing, the structurally stand-alone design of the electrode member 31D allows the majority of its volume to be made from more economical material for cost conservation. For instance, the base body 31-1D of the exemplary electrode member 31D may be substantially made of a relative inexpensive insulating material (e.g., glass or plastic), while only the sensing surface thereof being provided with conductive coating of sufficient thickness (e.g., a gold layer that possesses sufficiently low surface roughness and offers high compatibility for probe immobilization). Suitable material for the base body 31-1D may have a resistivity substantially greater than 10⁻⁶ ΩM. In some embodiments, the material for the base body 31-1D may include one or more of, e.g., semiconductor materials (which generally possess resistivity ranging from 10⁻⁶ to 10⁶ ΩM) and dielectric materials (which generally having resistivity ranging from 10¹¹ to 10¹⁹ ΩM). In some embodiments, the material used to form the base body 31-1D includes silicon substrate or glass substrate.

For another, as the surface modification process of the electrode member (e.g., immobilization of the bio-sensitive material such as ligand or antibody) is often temperature sensitive (e.g., cannot withstand high processing temperature that conventional semiconductor devices are normally subjected to), the structurally separated electrode member 31D further allows the capturing surface of the electrode member to be prepared independent of the substrate (e.g., PCB 19) or the micro sensor chip (e.g., chip member 32D) at a lower temperature processing environment.

To attain higher a degree of sensing quality, the conductive coating of the electrode member (e.g., coating layer 31-2D) may be formed by suitable thin film deposition technique (e.g., physical deposition such as electrode plating or sputtering) to ensure surface smoothness and layer uniformity. In some embodiments, a surface roughness of the coating layer 31-2D is kept substantially less than 10 μm. In some embodiments, a width of the conductive coating's pattern profile may vary along the length of the electrode. For instance, the region where the biosensor probes are immobilized may be provided with a greater width than the immediately upstream segment of the coating pattern profile.

The coating layer 31-2D may include one or more suitable conductive material arranged in thin foil/film, which may include, e.g., carbon cloth, carbon brush, carbon rod, carbon mesh, carbon veil, carbon paper, carbon felt, granular activated carbon, granular graphite, carbonized cardboard, graphite film, reticulated vitreous carbon, stainless steel sheet, stainless steel mesh, stainless steel scrubber, silver film, nickel film, copper film, gold film, and titanium film.

In the illustrated embodiment, a chip member 32D of the sensing device includes a sensor array 32-1D and a contact pad 32-2D. The sensor array 32-1D may include an array of interweaving doped regions and oxide regions, in which an array of source/drain and gate oxide regions of the bio-sensing elements are defined. In some embodiments, the bio-sensing elements comprise ion-sensing field effect transistors, (ISFET), which is a type of bio sensitive micro/nano semiconductor based device capable of detecting variation of ion concentration in a sample analyte. In some embodiments, the on chip sensor elements may include the source and drain regions of an extended gate device (EGFET), whose gate component is formed remotely at a separate location (e.g., over the coating layer 31-2D of the electrode member). The contact pad 32-2D is provided to serve as I/O interface between the chip member 32D and the substrate (e.g., substrate 19).

Although not explicitly observable from the instant illustration, a lower micro channel member made from a fluid sealing material (i.e., a material capable of forming a substantially fluid-tight interface upon assembly, e.g., layer 18 in FIG. 6)) is provided over the sensing surfaces of the electrode member 31D and the chip member 32D. As depicted previously, the lower micro channel member may comprise an elastic bulk material in which various micro-fluid channel features are defined. Among the embedded micro channel features are a reaction chamber 18-3D and an active chamber 18-4D formed in alignment with the capture surface of the electrode member and the active surface of the chip member, respectively, upon assembly. In addition, a locally elevated fluid passage (e.g., the suspended section 18-5D, which will be depicted in further detail in subsequent figures) is provided to enable fluid communication between sampling chambers 18-3D, 18-4D.

In the illustrated embodiments, an inlet 18-6D is formed toward one end of the reaction chamber 18-3D while the suspended section 18-5D is formed toward the other end thereof. The inlet 18-6D may be configured to enable fluid access from an upper level of the multi-deck micro channel structure (e.g., from the higher layer members 15-1, 15-2 as shown in FIG. 6). In some embodiments, another suspended section may be formed on one end (e.g., upstream end) of the active chamber 18-4D, while another outlet is provided on the other end (e.g., downstream end) of the active chamber to guide the spent reaction fluid toward a waste collection (e.g., chamber 18-1C shown in FIG. 7).

In some embodiments, the cross sectional dimension of the sampling chambers (e.g., the active chamber 18-4D and the reactive chamber 18-3D) are designed in accordance with a predetermined layout design rule. In some embodiments, the widths of the active chamber 18-4D and the reactive chamber 18-3D are substantially the same. In some embodiments, a channel length of the active chamber 18-4D along the sample flow path (i.e., the first chamber length) is substantially shorter than a channel length of the reaction chamber 18-3D (i.e., the second chamber length). In some embodiments, a ratio between the first chamber length and the second chamber length is substantially less than 1. In some embodiments, the ratio between the first and the second chamber length is in a range of about 1×10⁻⁴ to about 1.

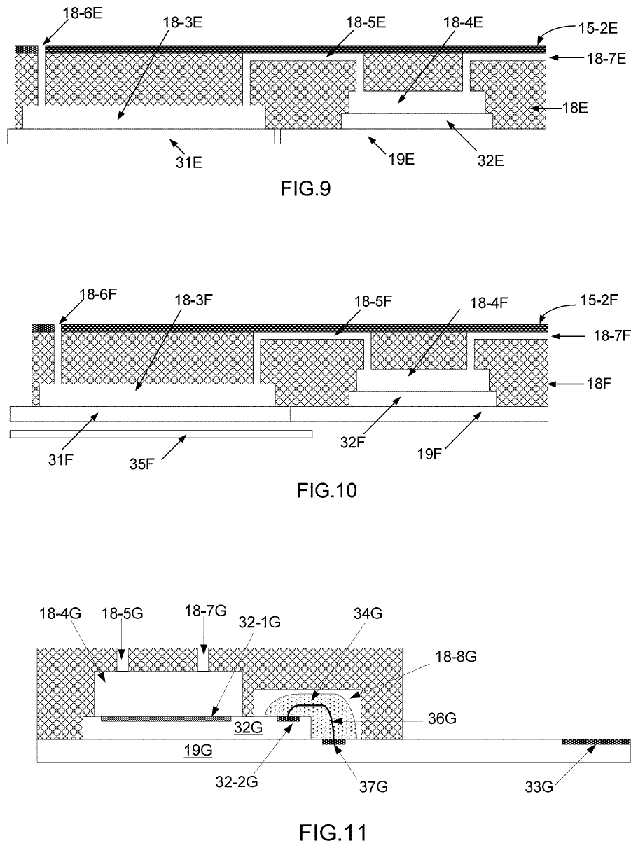

FIG. 9 illustrates a cross sectional view of a sensor cartridge in accordance with some embodiments of the instant disclosure along a cutline through the sampling chambers thereof. For instance, FIG. 9 illustrates a cross sectional view of a sensing device along cutline AA′ as shown in FIG. 7.

As can be better observed from this sectional view, the exemplary sensor cartridge has an electrode member 31E and a chip member 32E arranged at different elevation with respect to the mounting surface of the substrate 19E. For instance, in the illustrated embodiment, the active surface of the chip member 32E vertically closer a border layer 15-2E than the capture surface of the electrode member 31E. In some embodiments, the chip member 32E is disposed on the mounting face of a substrate 19E (e.g., onboard) while the electrode member 31E is disposed outside the mounting face of the substrate 19E (e.g., off board).

In the illustrated embodiment, the active surface of the chip member 32E is shorter vertical distance to a border layer 15-2E less than that of the capture surface of the electrode member 31E.) comes in contact with a portion of the chip member 32E and the electrode member 31E (e.g., the periphery/edge region), thereby forming a substantially fluid-tight sealing interface around the respective sampling surfaces of the chip member 32E and the electrode member 31E. For instance, the lower channel layer 18E internally defines a lower portion of the cartridge's embedded micro channel structure, which includes a reaction chamber 18-3E, an active chamber 18-4E, and a suspended section 18-5E arranged between the two sampling chambers to enable fluid access for the active surface and the capture surface from the micro channel structure. As schematically illustrated (e.g., in FIGS. 9-11), the sampling chambers 18-3E/F/G, 18-4E/F/G are provided with smaller planar dimension than the sensing surfaces of the sensor devices 31E/F/G, 32E/F/G, thereby allowing the lower channel layer 18E/F/G to establish a proper fluid seal around the periphery of the sensor components upon assembly.

In the illustrated embodiment, the exemplary suspended section 18-5E resembles an overpass bridge that connects the two sampling chambers at a raised elevation. For instance, the suspended section 18-5E extends to an elevation higher than an immediate upstream section thereof (e.g., being raised higher than the reaction chamber 18-E over the electrode member 31E). As illustrated by various embodiments, the micro-channel-structure defines an upstream (e.g., toward a sample collection inlet, such as port 11-2 shown in FIG. 4A) and a downstream direction (e.g., toward a waste collection chamber, such as chamber 18-1C shown in FIG. 7). While the exemplary electrode member 31E is shown to be arranged toward the upstream with respect to the chip member 32E, it should be noted that, depending on the operating principle of the bio-sensing device (e.g., ISFET), the sequential placement of the sampling surfaces may not be limited to the illustrated order.

The various micro channel structures in the lower channel layer 18E may be formed by embedded, semi-exposed channel features defined therein. For instance, the reaction and the active chambers 18-3E, 18-4E may be formed by recessed, downward facing troughs provided on the bottom face of the lower channel layer 18E, which, upon coupling with the electrode member 31E, form the enclosed sampling chambers. On the other hand, the exemplary suspended section 18-5E is formed by an inverted U-shape conduit feature that comprises a shallower horizontal trench segment (exposed toward the top surface of the lower channel layer 18E) and a pair of vertically traversing via segments having unequal length (e.g., depths) joined at the two ends of the horizontal segment. Upon placement of the border layer 15-2E over the lower channel layer 18E, the semi-opened trench feature of the suspended section 18-5E is sealed to form an enclosed portion of the micro channel structure. In some embodiments, the border layer 15-2E may be a layer of water resistant pad (e.g., double sided tape). In some embodiments, the border layer 15-2E may be part of the upper level package components (e.g., the bottom surface of the middle layer member 15-2 has shown in FIG. 4).

As further shown in the instant embodiment, lower level of the micro channel structure embedded in the lower channel layer 18E receives fluid input from the access port 18-6E. The micro channel structure subsequently guides the input fluid sequentially over the various sampling surfaces of the sensor device. The spent fluid may then exit the channel system through an extraction port 18-7E arranged downstream of the flow path.

FIG. 10 illustrates a cross sectional view of a sensor cartridge in accordance with some embodiments of the instant disclosure along a cutline through the sampling chambers thereof, e.g., along cutline AA′ as shown in FIG. 7.

While the majority of features illustrated in FIG. 10 share substantial resemblance with that shown in FIG. 9 (therefore are omitted for the brevity of disclosure), the exemplary embodiment of FIG. 10 is provided with a temperature control component 35F below the electrode member 31F. Depending on the type of sample analyte and its correspondingly preferred reaction environment condition, the temperature control component 35F may provide temperature regulation (e.g., heating/cooling) in the vicinity of the micro fluid flow path, thereby enhancing reaction efficiency over the bio-sensor components. In some embodiments, the temperature control component 35F is provided internally in the sensor cartridge, and may be operated through the acceptance of externally provided power. In some embodiments, the temperature control component 35F is provided externally off-board the sensor cartridge (e.g., arranged in a cartridge reader, such as reader 20 shown in FIG. 2).

In addition, as shown in the example of FIG. 10, in some embodiments, the electrode member 31F may be structurally connected to the substrate 19F. For instance, while a structurally separated electrode member offers additional packaging flexibility, in some embodiments, an electrode member (e.g., electrode 31F) may be provided on a designated region over the mounting surface of a substrate (e.g., an on-board region of a PCB provided with conductive coating) for the sake of overall structural simplicity and reduction of parts.

FIG. 11 illustrates a schematic cross sectional view showing an active chamber of a sensor cartridge according to some embodiments of the present disclosure. It should be noted that the schematic cutaway view is provided to show various features and their functional relationship, and does not necessarily reflect the actual sectional view along a particular cutline.

In the illustrated embodiment, the active chamber 18-4G is formed by a cavity feature defined in the lower channel layer (e.g., member 18 as shown in FIG. 6), which is arranged over the chip member 32G and the substrate 19G upon assembly. The lower channel layer 18G forms a substantially fluid-tight sealing interface around the chip member 32G over the substrate 19G.

In some embodiments, a plurality of contact pads 33G, 37G are formed on the substrate 19G. In some embodiments, the contact pad 37G is formed on a mounting face of the substrate 19G. An edge of the chip member 32G having contact pads 32-2G is positioned to be in alignment with the contact pad 37G. The contact pads 32-2G and 37G are electrically coupled to each other through wire bonding 36G. Further, an encapsulation 34G is disposed over the contact pads 32-2G, 37G, and the wire bonding 36G. In this way, the wire bonding 36G may be protected by the encapsulation 34G from environment hazard such as humidity or mechanical stress. Moreover, in the illustrated embodiment, the encapsulation 34G covers only one of the four edges of the chip member 32G. Accordingly, the remaining edges of the chip member 32G free from electrical bonding thus form a plurality of free edges. With the reduction of mechanical hindrance from the electrical interface, maximized fluid exposure/accessibility may be ensured between the chip member 32G and the micro channel structure (e.g., active chamber 18-4G).

During operation, fluid may enter the active chamber 18-4G through the suspended section 18-5G and exit the active chamber through the extraction port 18-7G. The fluid is guided over the active surface of the chip member 32G during the process. On the other hand, the lower channel layer provides fluid isolation between the sampling regions of the sensor chip 32G and the sensitive electrical components thereof. For instance, as may be observed from the instant illustration, only a selective portion of the sensor chip surface (e.g., the first sampling area 32-1G of the active surface) exposed within the active chamber 18-4G is accessible by the passing fluid.

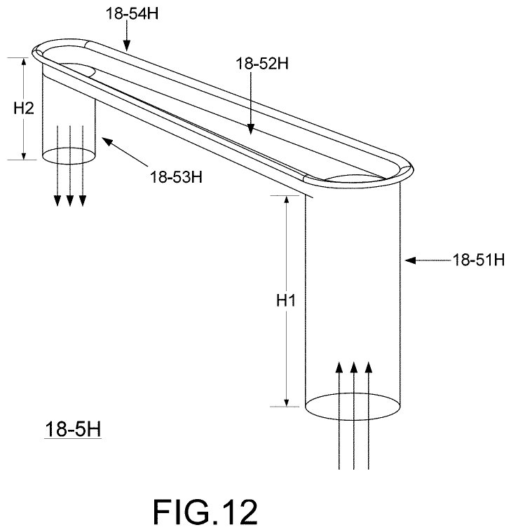

FIG. 12 provides a perspective illustration of a suspended section in the micro-fluidic channel structure of a sensor cartridge according to some embodiments of the present disclosure. For instance, FIG. 12 shows an isolated view of an exemplary suspended section to provide enhanced structural clarity.

As previously depicted, a suspended section (e.g., conduit feature 18-5H) is provided between a reaction chamber and an active chamber in the biosensor cartridge in accordance with the instant disclosure. In some embodiments, the suspended section 18-5H includes a first column section 18-5H1, a second column section 18-53H, and an overpass section 18-52H. The first column section 18-5H1 and the second column section 18-53H are formed respectively at the opposite ends of the overpass section 18-52H.

The overpass section 18-52H may be provided as a shallow trench feature (e.g., a blind-hole like recess) formed on an upward facing surface of a bulk component made from water resisting material (e.g., lower member layer 18 as shown in FIG. 6). In some embodiments, the semi-opened trench portion of the overpass section 18-52H is designed to sealed up upon engagement with an upper level package component of the sensor cartridge (e.g., middle layer member 15-2 as shown in FIG. 6). In some embodiments, the suspended section 18-5H may be provided with a seal ring 18-54H along a periphery region thereof to further enhance fluid sealing capability, thereby increasing device reliability.

As can be further observed from the schematic illustration, a length of the first column 18-5H1 (i.e., height H1) is different from (e.g., greater than) a length of the second column 18-53H (i.e., height H2). The height differentiation in the column sections 18-51H/18-53H enables additional flexibility in package layout design. For instance, such suspended channel arrangement enjoys fabrication simplicity while offering greater pliancy in the accommodation of step variations among different circuit components.

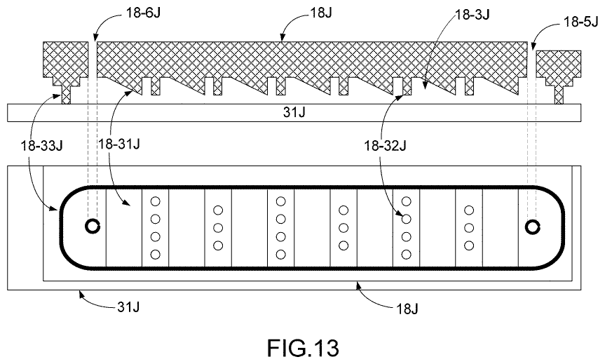

FIG. 13 illustrates a cross sectional view showing a reaction chamber of a sensor cartridge according to some embodiments of the present disclosure. The exemplary reaction chamber 18-3J may be formed upon placement of a lower channel member (e.g., layer 18J) over an electrode member 31J. A substantially fluid-tight seal is formed between layer 18J and the electrode member 31J. In some embodiments, a seal ring feature 18-33J is provided on layer 18 around a periphery of the reaction chamber 18-3J to ensure proper sealing along the components interface.

In some embodiments, inlet port 18-6J and extraction port 18-5J are formed at opposite ends of the reaction chamber 18-3J. In order to facilitate higher reaction efficiency, an inner surface of the micro channel structure exposed to the reaction chamber 18-3J may be provided with an agitating/turbulence inducing features. For instance, in the illustrated embodiment, an agitating surface is provided at the top (ceiling) of the reaction chamber 18-3J, with its protruding serration patterns arranged facing toward the capture surface of the electrode member 31J. The exemplary agitating surface includes a plurality of serrated agitators 18-31J and column agitators 18-32J, and traverses between the inlet port 18-6J and the extraction port 18-5J. The serrated agitators 18-31J and the protruding agitators 18-32J are interleavingly arranged along the length of the reaction chamber 18-3J. As further shown in the illustrated embodiments, the column agitators 18-32J in adjacent rows may be arranged in an interposingly offset pattern along the fluid flow direction.

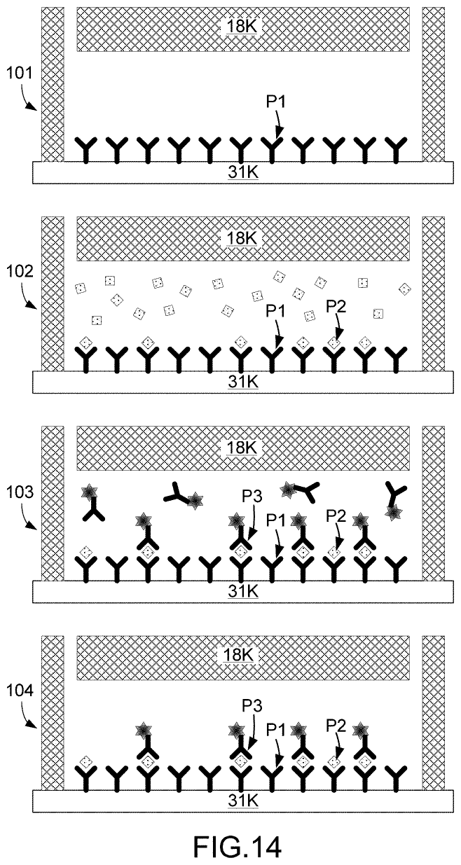

FIG. 14 illustrates an exemplary sample interaction in the flow path of a sensor cartridge according to some embodiments of the present disclosure. For instance, FIG. 14 illustrates an assay flow in a sensor cartridge according to some embodiments of the present disclosure. Particularly, FIG. 14 illustrates an exemplary embodiment of the assay process performed within the reaction chamber of the sensor cartridge.

A reaction chamber is formed between the lower channel layer 18K and the electrode member 31K. In some embodiments, an array of capture probes P1 is disposed over the capture surface of the electrode member 31K, as shown in process 101.

A sample fluid having target molecules P2 are then introduced into the reaction chamber. The capture probes P1 are configured to capture the target molecules P2 and affix the target molecules P2 to remain within the reaction chamber, as shown in process 102.

In some embodiments, a wash fluid is used to wash away target molecules P2 that were not captured by the capture probes P1. A reaction fluid having labeling probes P3 are then introduced into the reaction chamber. The capture probes P1 are configured to capture the target molecules P2 and affix the labeling probes P3 to remain within the reaction chamber, as shown in process 103.

A wash fluid is provided to wash away labeling probes P3 that were not captured by the target molecules P2, as shown in process 104.

In an exemplary embodiment, the capture probes P1, the target molecules P2, and the labeling probes P3 can respectively be a capture antibody, an antigen, and a primary antibody. The primary antibody is conjugated with a substance detectable by the sensing device.

In some embodiments, an initial readout from the sensing device is performed before starting the assay process. After the assaying process, a final readout from the sensing device is performed. The difference between the initial readout and the final readout is calculated to generate an output that reflects the concentration of the target molecules P2.

In some other embodiments, an initial readout from the sensing device is not needed. a final readout is measured to generate an output that reflect the concentration of the target molecules P2.

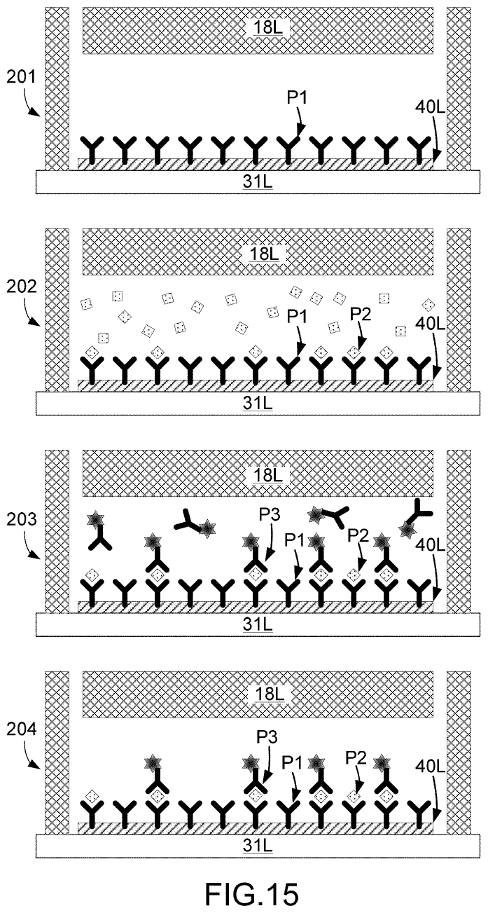

FIG. 15 illustrates an exemplary sample interaction in the flow path of a sensor cartridge according to some embodiments of the present disclosure. For instance, FIG. 15 illustrates an exemplary embodiment of the assay process performed within the reaction chamber of the sensor cartridge.

A reaction chamber is formed between the lower channel layer 18L and the electrode member 31L. In some embodiments, an array of capture probes P1 is disposed over the capture surface of the electrode member 31L, as shown in process 201.

In some embodiments, the capture probes P1 are arranged on a coating layer of the electrode member 31L. Further, a linking layer 40L is disposed between the capture probes P1 and the electrode member 31L. The linking layer 40L may enhance the retention of the capture probes P1. A sample fluid having target molecules P2 are then introduced into the reaction chamber.

The capture probes P1 are configured to capture the target molecules P2 and affix the target molecules P2 to remain within the reaction chamber, as shown in process 202.

In some embodiments, a wash fluid is used to wash away target molecules P2 that were not captured by the capture probes P1. The wash fluid can be a buffer fluid.

A reaction fluid having labeling probes P3 are then introduced into the reaction chamber. The target molecules P2 are configured to capture the target molecules P2 and affix the labeling probes P3 to remain within the reaction chamber, as shown in process 203.

A wash fluid is used to wash away labeling probes P3 that were not captured by the target molecules P2, as shown in process 204.

In an exemplary embodiment, the capture probes P1, the target molecules P2, and the labeling probes P3 can respectively be a capture antibody, an antigen, and a primary antibody. The primary antibody is conjugated with a substance detectable by the sensing device.

In some embodiments, an initial readout from the sensing device is performed before starting the assay process. After the assay process, a final readout from the sensing device is performed. The difference between the initial readout and the final readout is calculated to generate an output that reflects the concentration of the target molecules P2.

In some other embodiments, an initial readout from the sensing device is not needed. Rather, a final readout is measured to generate an output that reflect the concentration of the target molecules P2.

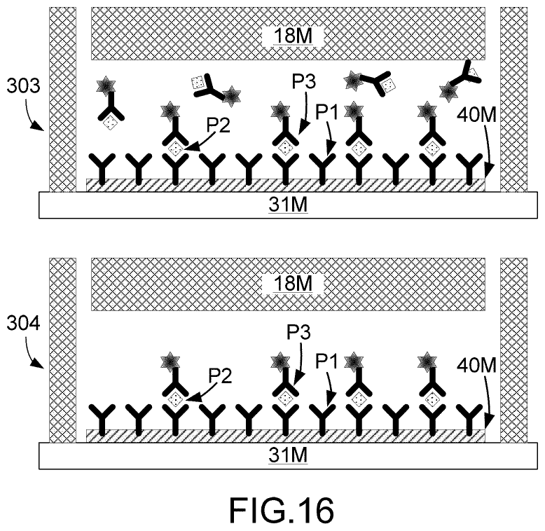

FIG. 16 illustrates an exemplary sample interaction in the flow path of a sensor cartridge according to some embodiments of the present disclosure. For instance, FIG. 16 illustrates an exemplary embodiment of the assay process performed within the reaction chamber of the sensor cartridge.

A reaction chamber is formed between the lower channel layer 18M and the electrode member 31M. In some embodiments, an array of capture probes P1 is disposed over the capture surface of the electrode member 31M. Further, in some other embodiments, a linking layer 40M is disposed between the capture probes P1 and the electrode member 31M. The linking layer 40M may enhance the retention of the capture probes P1. A sample fluid having target molecules P2 and labeling probes P3 affixed to each other is prepared. The sample fluid having the target molecules P2 and labeling probes P3 are then introduced into the reaction chamber. The target molecules P2 are captured by the capture probes P1 and configured to remain within the reaction chamber, as shown in process 303. A wash fluid is used to wash away excess sample fluid, as shown in process 304.

In some embodiments, the capture probes P1, the target molecules P2, and the labeling probes P3 can respectively be a capture antibody, an antigen, and a primary antibody. The primary antibody is conjugated with a substance detectable by the sensing device.

In some embodiments, an initial readout from the sensing device is performed before starting the assay process. After the assay process, a final readout from the sensing device is performed. The difference between the initial readout and the final readout is calculated to generate an output that reflects the concentration of the target molecules P2.

In some other embodiments, an initial readout from the sensing device is not needed. Rather, a final readout is measured to generate an output that reflect the concentration of the target molecules P2.

Accordingly, one aspect of the instant disclosure provides a sensor cartridge that comprises a sensing device, comprising a chip member comprising an active surface disposed over a mounting face of a substrate, the active surface defines a first sampling area; an electrode member comprising a capture surface, the capture surface defining a second sampling area; wherein the active surface of the chip member is arranged projectively offset the capture surface of the electrode member, wherein a ratio of the first sampling area and the second sampling area is substantially less than 1; and a micro-channel-structure arranged over the sensing device and configured to transport fluid to the active surface and the capture surface.

In some embodiments, the ratio between the first sampling area to the second sampling area is in a range of about 1×10⁻⁸ to about 1.

In some embodiments, the micro-channel structure is in contact with the chip member and the electrode member, and forms a substantially fluid-tight sealing interface therewith.

In some embodiments, the electrode member is a structurally separated member from the substrate.

In some embodiments, the electrode member is disposed outside the mounting face of a substrate

In some embodiments, the active surface of the chip member is arranged at a level different than the capture surface of the electrode member with respect to the mounting surface of the substrate.

In some embodiments, the electrode member further comprises a base body, and the capture surface comprises an array of probe immobilized over the base body, a material of the base body has a resistivity substantially greater than 10⁻⁶ ΩM.

In some embodiments, the electrode member further comprises a base body, and the capture surface comprises an array of probe immobilized over a coating layer on the base body, a surface roughness of the coating layer is substantially less than 10 μm.

In some embodiments, the micro-channel-structure includes a suspended section arranged between the active surface and the capture surface, the suspended section of the micro-channel-structure is arranged at an elevation higher than an immediate upstream section thereof.

In some embodiments, the chip member includes a microchip mounted with a plurality of free edges, the active surface is arranged on the microchip facing away the mounting surface of the substrate.

In some embodiments, the substrate includes an I/O interface arranged at an edge portion thereof.

Accordingly, another aspect of the instant disclosure provides a sensor cartridge that comprises a sensing device, comprising a chip member having an active surface disposed over a mounting face of a substrate; an electrode member having a capture surface; and a micro-channel-structure arranged over the sensing device and sequentially transport fluid over the capture surface and the active surface. The micro-channel-structure includes a suspended section arranged between the active surface and the capture surface.

In some embodiments, the micro-channel-structure defines an upstream and a downstream direction; the electrode member is arranged toward the upstream with respect to the chip member.

In some embodiments, the suspended section of the micro-channel-structure is arranged at an elevation higher than an immediate upstream section thereof.

In some embodiments, the micro-channel structure defines an active chamber having a first chamber length over the active surface and a reaction chamber having a second chamber length over the capture surface. The suspended section is arranged between the reaction chamber and the active chamber.

In some embodiments, a ratio between the first chamber length and the second chamber length is substantially less than 1.

In some embodiments, the ratio is in a range of about 1×10⁻⁴ to about 1.

In some embodiments, the reaction chamber of the micro-channel structure is provided with an agitating surface arranged facing the capture surface.

In some embodiments, the micro-channel structure has a planar coverage beyond the mounting surface of the substrate.

In some embodiments, the distance between the active surface and the capture surface no less than 0.1 mm.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A sensor cartridge, comprising:
a sensing device, comprising:
a chip member comprising an active surface disposed over a mounting face of a substrate, the active surface defines a first sampling area;
an electrode member comprising a capture surface, the capture surface defining a second sampling area;
wherein the active surface of the chip member is arranged projectively offset the capture surface of the electrode member,
wherein a ratio of the first sampling area and the second sampling area is substantially less than 1; and
a micro-channel-structure arranged over the sensing device and configured to transport fluid to the active surface and the capture surface.

2. The cartridge of claim 1, wherein
the ratio between the first sampling area to the second sampling area is in a range of about 1×10⁻⁸ to about 1.

3. The cartridge of claim 1,
wherein the micro-channel structure is in contact with the chip member and the electrode member, and forms a substantially fluid-tight sealing interface therewith.

4. The cartridge of claim 1, wherein
the electrode member is a structurally separated member from the substrate.

5. The cartridge of claim 4, wherein the electrode member is disposed outside the mounting face of a substrate

6. The cartridge of claim 4,
wherein the active surface of the chip member is arranged at a level different than the capture surface of the electrode member with respect to the mounting surface of the substrate.

7. The cartridge of claim 4,
wherein the electrode member further comprises a base body, and the capture surface comprises an array of probe immobilized over the base body,
wherein the base body comprises a material having resistivity substantially greater than 10⁻⁶ ΩM.

8. The cartridge of claim 4,
wherein the electrode member further comprises a base body, and the capture surface comprises an array of probe immobilized over a coating layer on the base body,
wherein a surface roughness of the coating layer is substantially less than 10 μm.

9. The cartridge of claim 1,
wherein the micro-channel-structure includes a suspended section arranged between the active surface and the capture surface,
wherein the suspended section of the micro-channel-structure is arranged at an elevation higher than an immediate upstream section thereof.

10. The cartridge of claim 1,
wherein the chip member includes a microchip mounted with a plurality of free edges,
wherein the active surface is arranged on the microchip facing away the mounting surface of the substrate.

11. The cartridge of claim 1,
wherein the substrate includes an I/O interface arranged at an edge portion thereof.

12. A sensor cartridge, comprising:
a sensing device, comprising:
a chip member having an active surface disposed over a mounting face of a substrate;
an electrode member having a capture surface; and
a micro-channel-structure arranged over the sensing device and sequentially transport fluid over the capture surface and the active surface,
wherein the micro-channel-structure includes a suspended section arranged between the active surface and the capture surface.

13. The cartridge of claim 12,
wherein the micro-channel-structure defines an upstream and a downstream direction;
wherein the electrode member is arranged toward the upstream with respect to the chip member.

14. The cartridge of claim 12,
wherein the suspended section of the micro-channel-structure is arranged at an elevation higher than an immediate upstream section thereof.

15. The cartridge of claim 12,
wherein the micro-channel structure defines an active chamber having a first chamber length over the active surface and a reaction chamber having a second chamber length over the capture surface,
wherein the suspended section is arranged between the reaction chamber and the active chamber.

16. The cartridge of claim 15,
wherein a ratio between the first chamber length and the second chamber length is substantially less than 1.

17. The cartridge of claim 16,
wherein the ratio is in a range of about 1×10⁻⁴ to about 1.

18. The cartridge of claim 15,
wherein the reaction chamber of the micro-channel structure is provided with an agitating surface arranged facing the capture surface.

19. The cartridge of claim 12,
wherein the micro-channel structure has a planar coverage beyond the mounting surface of the substrate.

20. The cartridge of claim 12,
wherein the distance between the active surface and the capture surface no less than 0.1 mm.
